(19) United States
(12) Patent Application Publication
(10) Pub. No.: US 2026/0232279 A1
(43) Pub. Date: Aug. 13, 2026

(54) SYSTEM AND METHOD FOR PROVIDING RECOMMENDATIONS TO PROMOTE SLEEP

(71) Applicant: BEDGEAR, LLC, Farmingdale, NY (US)

(72) Inventors: Eugene Alletto, JR., Glen Head, NY (US); Lorenzo Turicchia, Arlington, MA (US)

(73) Assignee: BEDGEAR, LLC, Farmingdale, NY (US)

(21) Appl. No.: 19/536,704

(22) Filed: Feb. 11, 2026

Related U.S. Application Data
(60) Provisional application No. 63/758,118, filed on Feb. 13, 2025.

(51) Int. Cl. A61B 5/00; A61B 5/08

(52) U.S. Cl. CPC A61B 5/7275; A61B 5/0816; A61B 5/4809; A61B 5/4815

(57) ABSTRACT

A system to promote sleep of a sleeper includes a support configured to support at least a portion of the sleeper's body. A first sensor is configured to be coupled to the sleeper's body and to monitor a first set of sleep parameters to provide a first data set based on the first set of sleep parameters. A second sensor is spaced apart from the sleeper's body and is configured to monitor a second set of sleep parameters to provide a second data set based on the second set of sleep parameters. A processor is configured to receive and analyze the data sets simultaneously and to execute an algorithm to provide a sleep recommendation based on the simultaneously analyzed data sets. A receiver is configured to receive the sleep recommendation and display information relating to the sleep recommendation. Methods of use are disclosed.

### TECHNICAL FIELD

The present disclosure generally relates to bedding, and more particularly to systems and methods to promote sleep, and more particularly, to systems and methods that collect and analyze a wide range of data to identify discrepancies between perceived and actual sleep experiences and/or provide recommendations for bedding, to enhance sleep quality and comfort.

### BACKGROUND

Sleep is critical for people to feel and perform their best, in every aspect of their lives. Sleep is an essential path to better health and reaching personal goals. Indeed, sleep affects everything from the ability to commit new information to memory to weight gain. It is therefore essential for people to use bedding that is comfortable in order to achieve restful sleep.

Systems have been developed that can be used at home to mimic certain aspects of a sleep study, such as, for example, a polysomnogram, by collecting certain data pertaining to a sleeper while the sleeper sleeps to help diagnose and treat sleep disorders. Some systems have incorporated sensors into various bedding, such as, for example, mattresses, mattress toppers, etc., to provide data pertaining to the bedding, such as, for example, temperature. The data pertaining to the sleeper and the data pertaining to the bedding are then analyzed separately to generate a sleep report. However, such systems do not analyze the data pertaining to the sleeper and the data pertaining to the bedding simultaneously using differential data to provide an accurate estimation of the sleeper's sleep experience. Furthermore, such systems do not recommend bedding, such as, for example, mattresses and/or pillows that are selected for the sleeper based on the sleeper's sleep report. This disclosure describes an improvement over these prior art technologies.

### SUMMARY

In one embodiment, in accordance with the principles of the present disclosure, a system to promote sleep of a sleeper comprises a support configured to support at least a portion of the sleeper's body. A first sensor is configured to be coupled to the sleeper's body and to monitor a first set of sleep parameters to provide a first data set based on the first set of sleep parameters. A second sensor is spaced apart from the sleeper's body and is configured to monitor a second set of sleep parameters to provide a second data set based on the second set of sleep parameters. A processor is configured to receive and analyze the data sets simultaneously and to execute an algorithm to provide a sleep product recommendation (also referred to herein as a sleep recommendation) based on the simultaneously analyzed data sets. A receiver configured to receive the sleep product recommendation and display information relating to the sleep recommendation.

In some embodiments, the sleep product recommendation includes suggestions for bedding based on the simultaneously analyzed data sets. In some embodiments, the bedding is selected from the group consisting of mattresses and pillows.

In some embodiments, analyzing the data sets simultaneously includes using differential measurements to compare differences between the data sets and determine the sleep product recommendation. In some embodiments, the sleep product recommendation includes suggestions for bedding based on the simultaneously analyzed data sets. In some embodiments, the bedding includes mattresses and pillows. In some embodiments, the sleep recommendation provides an accurate estimation of temperature and humidity conditions that are experienced by the sleeper during a sleep session. In some embodiments, the sleep recommendation includes suggestions for environmental adjustments. In some embodiments, the sleep recommendation identifies discrepancies between perceived and actual sleep experiences.

In some embodiments, the first set of sleep parameters includes sleep movements of the sleeper during a sleep session and sleep positions of the sleeper during the sleep session. In some embodiments, the sleep movements record the frequency of the sleeper tossing and turning during the sleep session. In some embodiments, the sleep movements record the sleeper's movement patterns. In some embodiments, the sleep positions monitor sleep positions assumed by the sleeper during the sleep session.

In some embodiments, the first set of sleep parameters includes localized temperature adjacent to the support. In some embodiments, the first set of sleep parameters includes localized humidity adjacent to the support.

In some embodiments, the first sensor is configured to be coupled to at least one of the sleeper's torso, the sleeper's back and the sleeper's stomach. In some embodiments, the first sensor is configured to be spaced apart from the support.

In some embodiments, the second set of sleep parameters includes at least one of a temperature of the support, a humidity of the support, an ambient temperature, an ambient humidity and an ambient luminosity. In some embodiments, the second set of sleep parameters includes a temperature of the support, a humidity of the support, an ambient temperature, an ambient humidity and an ambient luminosity.

In some embodiments, the second sensor is spaced apart from the support and the sleeper. In some embodiments, the support is a cushion. In some embodiments, the support is selected from the group consisting of a mat, a mattress topper and a mattress. In some embodiments, the support is a mattress.

In some embodiments, the system further comprises a computer, the computer including the processor and the receiver. In some embodiments, the system further comprises a smartphone, the smartphone including the processor and the receiver.

In one embodiment, in accordance with the principles of the present disclosure, a system to promote sleep of a sleeper comprises a support configured to support at least a portion of the sleeper's body. A first sensor is configured to be coupled to the sleeper's body and to monitor a first set of sleep parameters to provide a first data set based on the first set of sleep parameters. A second sensor is spaced apart from the sleeper's body and is configured to monitor a second set of sleep parameters to provide a second data set based on the second set of sleep parameters. A third sensor is configured to be coupled to the sleeper's body and to track biometric data pertaining to the sleeper. A processor is configured to receive and analyze the data sets simultaneously and to execute an algorithm to provide a sleep recommendation based on the simultaneously analyzed data sets. A receiver is configured to receive the sleep recommendation and display information relating to the sleep recommendation.

In some embodiments, the sleep recommendation includes suggestions for bedding based on the simultaneously analyzed data sets. In some embodiments, the bedding is selected from the group consisting of mattresses and pillows.

In some embodiments, analyzing the data sets simultaneously includes using differential measurements to compare differences between the data sets and determine the sleep recommendation. In some embodiments, the sleep recommendation includes suggestions for bedding based on the simultaneously analyzed data sets. In some embodiments, the bedding includes mattresses and pillows. In some embodiments, the sleep recommendation provides an accurate estimation of temperature and humidity conditions that are experienced by the sleeper during a sleep session. In some embodiments, the sleep recommendation includes suggestions for environmental adjustments. In some embodiments, the sleep recommendation identifies discrepancies between perceived and actual sleep experiences.

In some embodiments, the system further comprises a covering that covers a portion of the support. The sleeper is configured for positioning in a space between the support and the covering. The first set of sleep parameters includes localized conditions within the space. In some embodiments, the localized conditions include temperature and humidity. In some embodiments, the localized conditions include fluctuations in temperature and/or humidity.

In some embodiments, the first set of sleep parameters includes sleep movements of the sleeper during a sleep session and sleep positions of the sleeper during the sleep session. In some embodiments, the sleep movements record a frequency of the sleeper tossing and turning during the sleep session. In some embodiments, the sleep movements record the sleeper's movement patterns. In some embodiments, the sleep positions monitor sleep positions assumed by the sleeper during the sleep session.

In some embodiments, the first sensor is configured to be coupled to at least one of the sleeper's torso, the sleeper's back and the sleeper's stomach. In some embodiments, the first sensor is configured to be spaced apart from the support.

In some embodiments, the second set of sleep parameters includes an ambient temperature, an ambient humidity and an ambient luminosity. In some embodiments, the second sensor is spaced apart from the support and the sleeper.

In some embodiments, the biometric data includes at least one of the group selected from heart rate variability, sleep stages, skin temperature and blood oxygen saturation. In some embodiments, the biometric data is acquired by tracking light, deep and REM sleep stages of the sleeper during a sleep session. In some embodiments, the biometric data is acquired by tracking the sleeper's heart rate variability such that the sleep recommendation provides insights into the sleeper's stress levels and recovery ability. In some embodiments, the biometric data is acquired by tracking the sleeper's oxygen levels. In some embodiments, the biometric data is acquired by tracking the sleeper's surface temperature variations.

In some embodiments, the sleep recommendation provides a sleep score. In some embodiments, the sleep score provides a summary metric based on heart rate variability, sleep stages, skin temperature and blood oxygen saturation to evaluate overall sleep quality.

In some embodiments, the system further comprises a smartwatch, the smartwatch including the third sensor. In some embodiments, the system the processor analyzes the data sets simultaneously using artificial intelligence to identify trends and pinpoint sources of discomfort.

In one embodiment, in accordance with the principles of the present disclosure, a method to promote sleep of a sleeper comprises: providing a support within an area; positioning at least a portion of the sleeper's body upon the support; coupling a first sensor to the sleeper's body such that the first sensor monitors a first set of sleep parameters and provides a first data set based on the first set of sleep parameters; positioning a second sensor within the area such that the second sensor is spaced apart from the sleeper's body, monitors a second set of sleep parameters and provides a second data set based on the second set of sleep parameters; providing a processor that receives and analyzes the data sets simultaneously and executes an algorithm to provide a sleep recommendation based on the simultaneously analyzed data sets; and providing a receiver that receives the sleep recommendation and displays information relating to the sleep recommendation.

In some embodiments, the sleep recommendation includes suggestions for bedding based on the simultaneously analyzed data sets. In some embodiments, the bedding is selected from the group consisting of mattresses and pillows.

In some embodiments, the data sets are analyzed simultaneously using differential measurements to compare differences between the data sets and determine the sleep recommendation. In some embodiments, the sleep recommendation includes suggestions for bedding based on the simultaneously analyzed data sets. In some embodiments, the bedding includes mattresses and pillows.

In some embodiments, the sleep recommendation provides an accurate estimation of temperature and humidity conditions that are experienced by the sleeper during a sleep session. In some embodiments, the sleep recommendation includes suggestions for environmental adjustments. In some embodiments, the sleep recommendation identifies discrepancies between perceived and actual sleep experiences.

In some embodiments, the first set of sleep parameters includes sleep movements of the sleeper during a sleep session and sleep positions of the sleeper during the sleep session. In some embodiments, the sleep movements record the frequency of the sleeper tossing and turning during the sleep session. In some embodiments, the sleep movements record the sleeper's movement patterns. In some embodiments, the sleep positions monitor sleep positions assumed by the sleeper during the sleep session.

In some embodiments, the first set of sleep parameters includes localized temperature adjacent to the support. In some embodiments, the first set of sleep parameters includes localized humidity adjacent to the support.

In some embodiments, the first sensor is configured to be coupled to at least one of the sleeper's torso, the sleeper's back and the sleeper's stomach. In some embodiments, the first sensor is configured to be spaced apart from the support.

In some embodiments, the second set of sleep parameters includes at least one of a temperature of the support, a humidity of the support, an ambient temperature, an ambient humidity and an ambient luminosity. In some embodiments, the second set of sleep parameters includes a temperature of the support, a humidity of the support, an ambient temperature, an ambient humidity and an ambient luminosity.

In some embodiments, the second sensor is spaced apart from the support and the sleeper. In some embodiments, the support is a cushion. In some embodiments, the support is selected from the group consisting of a mat, a mattress topper and a mattress. In some embodiments, the support is a mattress.

In some embodiments, the system further comprises a computer, the computer including the processor and the receiver. In some embodiments, the system further comprises a smartphone, the smartphone including the processor and the receiver.

In one embodiment, in accordance with the principles of the present disclosure, a method to promote sleep of a sleeper comprises: providing a support in an area; providing a covering that covers at least a portion of the support; positioning the sleeper in a space between the support and the covering; coupling a first sensor to the sleeper's body to monitor a first set of sleep parameters and provide a first data set based on the first set of sleep parameters; positioning a second sensor such that the second sensor is spaced apart from the sleeper's body, monitors a second set of sleep parameters and provides a second data set based on the second set of sleep parameters; coupling a third sensor to the sleeper's body to track biometric data pertaining to the sleeper; providing a processor that receives and analyzes the data sets simultaneously and executes an algorithm to provide a sleep recommendation based on the simultaneously analyzed data sets; and providing a receiver that receives the sleep recommendation and displays information relating to the sleep recommendation.

In some embodiments, the sleep recommendation includes suggestions for bedding based on the simultaneously analyzed data sets. In some embodiments, the bedding is selected from the group consisting of mattresses and pillows.

In some embodiments, analyzing the data sets simultaneously includes using differential measurements to compare differences between the data sets and determine the sleep recommendation. In some embodiments, the sleep recommendation includes suggestions for bedding based on the simultaneously analyzed data sets. In some embodiments, the bedding includes mattresses and pillows. In some embodiments, the sleep recommendation provides an accurate estimation of temperature and humidity conditions that are experienced by the sleeper during a sleep session. In some embodiments, the sleep recommendation includes suggestions for environmental adjustments. In some embodiments, the sleep recommendation identifies discrepancies between perceived and actual sleep experiences.

In some embodiments, the first set of sleep parameters includes localized conditions within the space. In some embodiments, the localized conditions include temperature and humidity. In some embodiments, the localized conditions include fluctuations in temperature and/or humidity.

In some embodiments, the first set of sleep parameters includes sleep movements of the sleeper during a sleep session and sleep positions of the sleeper during the sleep session. In some embodiments, the sleep movements record a frequency of the sleeper tossing and turning during the sleep session. In some embodiments, the sleep movements record the sleeper's movement patterns. In some embodiments, the sleep positions monitor sleep positions assumed by the sleeper during the sleep session.

In some embodiments, the first sensor is coupled to one of the sleeper's torso, the sleeper's back and the sleeper's stomach. In some embodiments, the first sensor is spaced apart from the support.

In some embodiments, the second set of sleep parameters includes an ambient temperature of the area, an ambient humidity of the area and an ambient luminosity of the area. In some embodiments, the second set of sleep parameters includes an ambient temperature of the area, an ambient humidity of the area and an ambient luminosity of the area. In some embodiments, the second sensor is spaced apart from the support and the sleeper.

In some embodiments, the biometric data includes at least one of the group selected from heart rate variability, sleep stages, skin temperature and blood oxygen saturation. In some embodiments, the biometric data is acquired by tracking light, deep and REM sleep stages of the sleeper during a sleep session. In some embodiments, the biometric data is acquired by tracking the sleeper's heart rate variability such that the sleep recommendation provides insights into the sleeper's stress levels and recovery ability. In some embodiments, the biometric data is acquired by tracking the sleeper's oxygen levels. In some embodiments, the biometric data is acquired by tracking the sleeper's surface temperature variations.

In some embodiments, the sleep recommendation provides a sleep score. In some embodiments, the sleep score provides a summary metric based on heart rate variability, sleep stages, skin temperature and blood oxygen saturation to evaluate overall sleep quality.

In some embodiments, the system further comprises a smartwatch, the smartwatch including the third sensor. In some embodiments, the processor analyzes the data sets simultaneously using artificial intelligence to identify trends and pinpoint sources of discomfort.

### BRIEF DESCRIPTION OF THE DRAWINGS

The present disclosure will become more readily apparent from the specific description accompanied by the following drawings, in which:

FIG. 1 is a perspective view, in part phantom, showing one embodiment of a system configured collect and analyze a wide range of data to identify discrepancies between perceived and actual sleep experiences and/or provide recommendations for bedding, to enhance sleep quality and comfort, in accordance with the principles of the present disclosure;

FIG. 2 is a schematic diagram showing one embodiment of a component of the system shown in FIG. 1, in accordance with the principles of the present disclosure;

FIG. 3 is a schematic diagram showing one embodiment of a component of the system shown in FIG. 1, in accordance with the principles of the present disclosure;

FIG. 4 is a schematic diagram showing one embodiment of a component of the system shown in FIG. 1, in accordance with the principles of the present disclosure; and

FIG. 5 is a flow diagram showing operation of one embodiment of the system shown in FIG. 1, in accordance with the principles of the present disclosure.

Like reference numerals indicate similar parts throughout the figures.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, conditions or parameters described and/or shown herein, and that the terminology used herein is for the purpose of describing particular embodiments by way of example only and is not intended to be limiting of the claimed disclosure.

A system is disclosed herein that is configured to collect and analyze a wide range of data to identify discrepancies between perceived and actual sleep experiences and/or provide recommendations for sleep products, to enhance sleep quality and comfort. As discussed below, the system uses “differential measurements”, rather than merely capturing disparate sensor data. The differential data can be used to provide a more accurate estimation of a sleeper's sleep quality and/or sleep efficiency and then optimize a sleep product (e.g., a mattress, a mattress topper, a pillow, etc.) based on the estimation of the sleeper's sleep quality and/or sleep efficiency, as discussed herein. Herein, a sleeper refers to an individual who is attempting to fall asleep, sleeping, or, and sleeping refers to the periods of time of a sleeper attempting to fall asleep, sleeping and awaking.

The system can include two or more separate and distinct sensors. In some embodiments, the system can include two sensors that are configured to measure temperature, humidity, light, the user's sleeping position, etc. The two sensors are used simultaneously. In some embodiments, a first one of the sensors is worn by the sleeper (on the body or on a sleep garment) and a second one of the sensors is placed at or near the bed to monitor the surrounding environmental conditions. In some embodiments, the first sensor can include a temperature sensor, a humidity sensor, a body position tracker and a movement detector. In some embodiments, the second sensor can include a temperature sensor, a humidity sensor and a light sensor.

The two sensors allow the system to measure sleeper movements to accurately record the frequency of tossing and turning during sleep. The two sensors further allow the system to track body positions to precisely monitor the various positions assumed by the sleeper throughout a period. Further, the two sensors allow the system to monitor environmental conditions, which allows the system to measure temperature and humidity both under the sheets, or bedding (microenvironment) and in the room, or area surrounding the sleeper (macroenvironment). Still further, the two sensors allow the system to optimize sleep products by utilizing differential measurements to enhance predictions for the optimal sleep products tailored to the individual sleeper.

The two sensors allow the system to provide accurate body position tracking. In particular, one of the sensors can be positioned on the torso, back, or belly of the sleeper to allow for precise monitoring of the sleeper's body position throughout a period. It is noted that to achieve precise monitoring of the sleeper's body position throughout a period, that a sensor should be attached or otherwise coupled to the sleeper's core, such as, for example, the part of the sleeper's body located between the pelvic floor and the diaphragm, to allow the sensor to track movement of the sleeper's core, rather than the sleeper's limbs or hands. Indeed, it is further noted that wrist-worn fitness and activity tracking devices such as Apple Watch, Whoop, or Garmin may be functional to track movement of the sleeper's hands but are incapable of accurately tracking movement of the sleeper's core. As such, sensors for the sleeper's torso, back and belly are both structurally and functionally distinct from wrist-worn devices. The two sensors enable differential measurements of the micro and macro environments, thus providing a more accurate estimation of the temperature and humidity experienced by the sleeper.

In some embodiments, the system can include three sensors and is configured for collecting sleep metrics and generating analytics or reports to assist sleep advisers of sleepers in fitting a sleep product (e.g., mattresses, pillows, etc.) such that the sleep product is tailored to individual sleep needs. In particular, by leveraging advanced monitoring devices, subjective feedback, and Artificial Intelligence (AI), the system collects and analyzes a wide range of data to identify discrepancies between perceived and actual sleep experiences. The result is actionable insights that empower sleep advisers to make precise suggestions for pillows, mattresses, and environmental adjustments, ultimately enhancing sleep quality and comfort.

In some embodiments, a first one of the three sensors is a micro-environment wearable sensor that collects data on body positions, movement patterns, and the localized temperature and humidity beneath the sheets or bedding. For example, the first sensor can be used to collect data regarding localized temperature and humidity in a space between a mattress and a sheet/blanket that is occupied by the sleeper. In some embodiments, a second one of the three sensors is a macro-environment sensor that is configured to be positioned in the user's sleeping area (e.g., the room or other area in which the sleeper's bed is positioned) such that the second sensor monitors ambient room conditions, such as, for example, temperature, humidity, and brightness. In some embodiments, a third one of the three sensors is a fitness and activity tracker, e.g., a smart watch, a fitbit, etc. or comparable device that is configured to track physiological parameters, including, for example, heart rate variability (HRV), sleep stages, skin temperature, and blood oxygen saturation (Sp0₂). As would be appreciated by one of ordinary skill in the art, the three separate and distinct sensors contribute essential data to a comprehensive profile of the sleeper's sleep comfort, allowing a processor to execute a program, such as, for example, a program that uses artificial intelligence to analyze the data collected by the three sensors to identify trends, pinpoint sources of discomfort, and generate tailored recommendations for use by a sleep adviser or the sleepers themselves.

In some embodiments, the first sensor of the three sensors is a micro-environment wearable sensor configured to collect data relating to microclimate temperature and humidity. In particular, the first sensor tracks localized conditions beneath the sheets or bedding. In some embodiments, fluctuations in temperature or sustained high humidity levels are correlated with sleeper movement patterns to assess if the bedding materials are contributing to discomfort. The first sensor may be configured to detect body orientation and movement patterns and record body positions, transitions, and sudden movements throughout a period. In some embodiments, frequent movements or high rates of positional changes are strong indicators of restlessness, often linked to environmental or bed-related factors. Additionally, the positions assumed by the sleeper can significantly differ from their self-reported descriptions to a sleep adviser. Understanding these discrepancies can be beneficial for accurate sleep product recommendations, as body position plays a key role in determining comfort and support requirements.

In some embodiments, the second sensor of the three sensors is a macro-environment sensor that is configured to detect ambient temperature and humidity. In some embodiments, the second sensor monitors the area's thermal and humidity trends since large fluctuations in these parameters are linked to environmental discomfort and sleep disruptions. In some embodiments, the second sensor captures variations in light intensity, assessing how light disturbances affect sleep patterns and comfort.

In some embodiments, the third sensor of the three sensors is a fitness and activity tracker, e.g. smart watch, a fitbit, etc. or comparable device that is configured to track light, deep, and REM sleep stages. In some embodiments, the third sensor is configured to monitor heart rate variability (HRV) to provide insights into the sleeper's stress levels and recovery ability during sleep, since changes in HRV may signal discomfort or environmental disruptions. In some embodiments, the third sensor is configured to monitor blood oxygen saturation (Sp0₂) by tracking the sleeper's oxygen levels, with dips potentially pointing to respiratory or environmental issues. In some embodiments, the third sensor is configured to monitor the sleeper's skin temperature by monitoring surface temperature variations. In some embodiments, the third sensor is configured to provide a sleep score, which includes a summary or calculated metric based on sleep stages, HRV, and movement data to evaluate overall sleep quality.

In some embodiments, the system may collect subjective feedback through structured questionnaires in which the sleeper is asked about their perceived comfort, personal preferences, and overall sleep satisfaction. The system can include a processor that is configured to simultaneously analyze differential data collected by the three sensors. The processor can execute a program having an algorithm, such as, for example, a program that uses artificial intelligence and serves as an analytical engine that processes data from all three of the sensors, as well as data from the questionnaires. The program synthesizes environmental, physiological, and behavioral data into actionable insights. These insights allow sleep advisers or sleepers to detect specific sources of discomfort, such as improper sleep product support or unsuitable environmental conditions. These insights can also allow advisers or sleepers to identify discrepancies between perceived and actual sleep behaviors. Furthermore, these insights can also allow sleep advisers to generate tailored suggestions for sleep product materials, including mattress type/firmness, pillow type/dimensions, and environmental adjustments. In some embodiments, the analytics or reports generated by the program can be presented in user-friendly formats, including timeseries graphs, charts, and comparative analysis tables, to assist sleep advisers in making data-driven decisions.

In some embodiments, the system can be adapted to provide tailored recommendations for sleep product fit. Indeed, by combining environmental, physiological, and subjective data, the system provides sleep advisers or sleepers with a comprehensive understanding of the user's sleep habits and comfort needs. Recommendations focus on sleep product materials to recommend an optimal mattress type/firmness and pillow types/dimensions that are tailored to the sleeper. Combining environmental, physiological, and subjective data also allows for environmental adjustments by providing recommendations for controlling temperature, humidity, and light conditions in order to create an ideal sleep environment. It is envisioned that this personalized approach provides comprehensive, data-driven analytics or reports, that enable sleep advisers to make optimal suggestions that help sleepers achieve maximum comfort and restorative sleep.

As used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

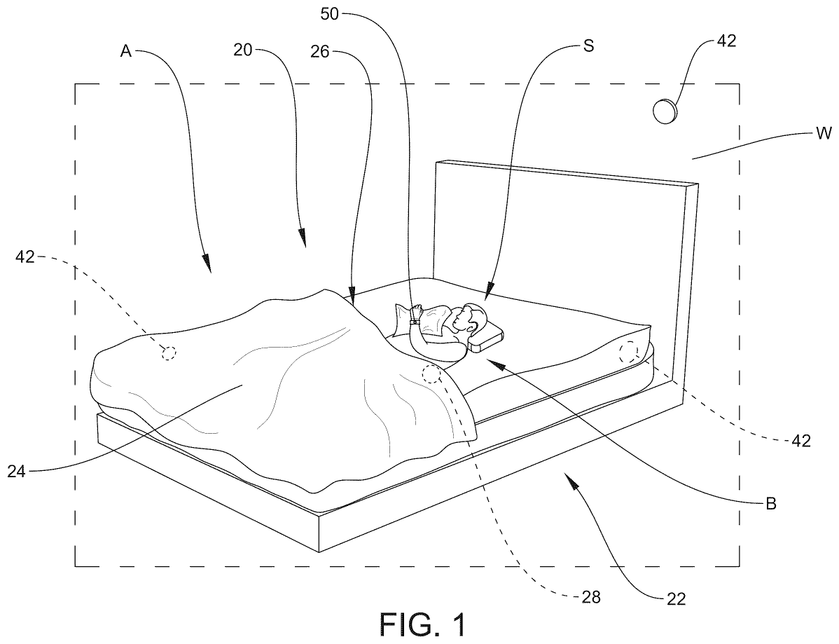

Reference will now be made in detail to the exemplary embodiments of the present disclosure, which are illustrated in the accompanying figures. Alternate embodiments are also disclosed. Turning to FIGS. 1-7, there are illustrated aspects of a system 20 that collects and analyzes a wide range of data to identify discrepancies between perceived and actual sleep experiences and/or provide recommendations for sleep products, to enhance sleep quality and comfort, in accordance with the principles of the present disclosure.

System 20 includes a support 22 that is configured to be positioned within an area A, such as, for example, a room of a building or other structure, as shown in FIG. 1. Support 22 is configured to support at least a portion of a body B of a sleeper S. In some embodiments, support 22 can include a mattress base or foundation, an adjustable mattress base, a mattress, a mattress topper, a mattress cover, a mattress protector, and combinations thereof. System 20 can include a cover 24, such as, for example, a sheet, blanket, comforter, quilt, etc. that is configured to cover at least a portion of sleeper S's body B when sleeper S's body is positioned on support 22. As shown in FIG. 1, at least a portion of sleeper S's body is positioned within a space 26 formed between the support 22 and the cover 24. As discussed herein, space 26 can define a microenvironment having a localized temperature and/or a localized humidity that may differ from an ambient temperature of area A and/or an ambient humidity of area A. The microenvironment defined by space 26 is thus distinguishable from the microenvironment defined by area A. Indeed, because, among other things, sleeper S's body temperature, sweating, etc. can increase the temperature and/or humidity within the microenvironment defined by space 26 such that the temperature and/or humidity within the microenvironment defined by space 26 is greater than the temperature and/or humidity within the macroenvironment defined by area A. System 20 is configured to detect such differences, which are taken into account when generating a sleep product recommendation, for example, as discussed herein.

System 20 includes a first sensor 28 that is configured to be coupled to the sleeper's body or the sleeper's garment and to monitor a first set of sleep parameters to provide a first data set based on the first set of sleep parameters. In some embodiments, first sensor 28 is configured to be coupled directly to sleeper S's body B. For example, first sensor 28 can be configured to be coupled directly to sleeper S's torso, back, or stomach. In some embodiments, first sensor 28 can be coupled directly to sleeper S's body B using tape and/or adhesive. For example, in some embodiments, first sensor 28 can be coupled directly to sleeper S's skin (on or underneath). In some embodiments, first sensor 28 can be worn by sleeper S, as shown in FIG. 3. For example, first sensor 28 can be positioned within a pocket of sleeper S's sleepwear, such as, for example, sleeper S's pajamas. As another example, first sensor 28 can be attached on sleeper S's sleepwear. As another example, first sensor 28 can be formed integral with the material that makes up sleeper S's sleepwear. As another example, first sensor 28 can be part of smart fabric that forms S's sleepwear. By coupling first sensor 28 to sleeper S's body or the sleeper S's garment, first sensor 28 is spaced apart from support 22.

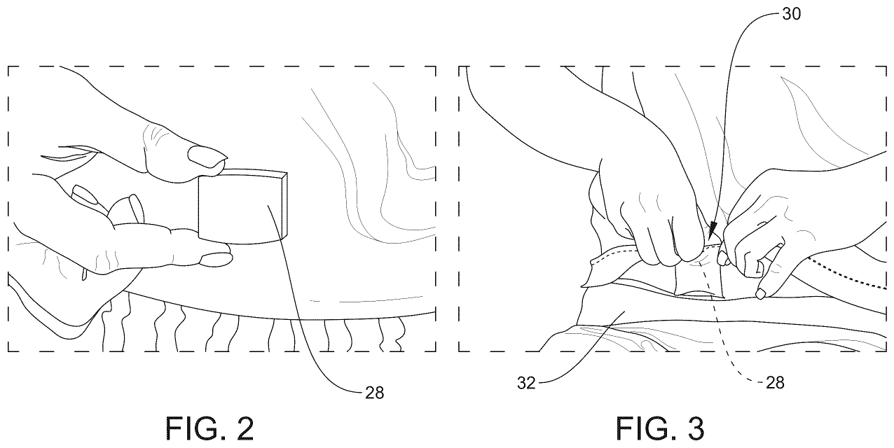

In some embodiments, first sensor 28 may include a plurality of different sensors, such as, for example, a temperature sensor 34, a humidity sensor 36, a body position tracker 38 and a movement detector 40, as shown in FIG. 2 and any combination thereof. First sensor 28 is configured to be positioned within the microenvironment defined by space 26 such that first sensor 28 can detect and record data pertaining to the temperature within the microenvironment using temperature sensor 34 and/or the humidity within the microenvironment using humidity sensor 36. In some embodiments, first sensor 28 can also be configured to detect and record data pertaining to body positions of sleeper S during a period and sleep movements of sleeper S during the period using body position tracker 38 and movement detector 40. In some embodiments, the sleep movements record a frequency of sleeper S, respectively tossing and turning during the period. In some embodiments, the sleep movements record sleeper S's movement patterns. In some embodiments, the body position tracker 38 monitors body position assumed by the sleeper during the period.

System 20 includes a second sensor 42 that is spaced apart from the sleeper S's body B and is configured to monitor a second set of sleep parameters to provide a second data set based on the second set of sleep parameters. In some embodiments, second sensor 42 can be positioned within area A such that second sensor 42 is spaced apart from support 22. That is, in some embodiments, second sensor 42 is not inserted within support 22 and is not coupled or otherwise attached to support 22. For example, in some embodiments, second sensor 42 is coupled to a wall W that defines a portion of area A, as shown in FIG. 1. In other embodiments, second sensor 42 may be coupled to support 22. For example, second sensor 42 may be inserted/embedded within support 22 and/or otherwise coupled or attached to support 22, as shown in FIG. 1. It is envisioned that system 20 can include a plurality of second sensors 42 that are positioned in different locations. For example, in one embodiment, a first one of second sensors 22 is coupled to wall W or another structure (e.g., floor, ceiling) that defines a portion of area A, and a second one of second sensors 42 is inserted/embedded within support 22 and/or otherwise coupled or attached to support 22.

In some embodiments, second sensor 42 may include a plurality of different sensors, such as, for example, a temperature sensor 44, a humidity sensor 46 and a light sensor 48, as shown in FIG. 3, or any combination thereof. Second sensor 42 is configured to be positioned within the macroenvironment defined by area A such that second sensor 42 can detect and record data pertaining to the temperature within the macroenvironment using temperature sensor 44, the humidity within the macroenvironment using humidity sensor 46 and/or the brightness within the macroenvironment using light sensor 48. Accordingly, in some embodiments, the second set of sleep parameters includes an ambient temperature, an ambient humidity and an ambient brightness such that second sensor 42 is capable of monitoring environmental conditions within area A, which may be different than conditions (e.g., temperature and/or humidity) within space 26. That is, while first sensor 28 can monitor conditions within space 26, second sensor 42 can monitor conditions outside of space 26 to allow system 20 to simultaneously monitor environmental conditions inside and outside of space 26 using first sensor 28 and second sensor 42 and to compare data collected by first sensor 28 and second sensor 42, as discussed here (i.e., “differential data”).

In some embodiments, the only sensors included in system 20 are first sensor 28 and second sensor 42. In other embodiments, system 20 includes a third sensor 50 in addition to first sensor 28 and second sensor 42. Third sensor 50 is configured to be coupled to the sleeper's body and to track biometric data pertaining to the sleeper. In some embodiments, third sensor 50 is configured to be worn around sleeper S's wrist, as shown in FIG. 1. Third sensor 50 may be a fitness and activity tracker. e.g., a smart watch, a fit bit device, or comparable device. In some embodiments, the biometric data that is tracked using third sensor 50 includes at least one of the group selected from heart rate variability, sleep stages, skin temperature and blood oxygen saturation. In some embodiments, the biometric data is acquired by third sensor 50 by tracking light, deep and REM sleep stages of sleeper S during a period. In some embodiments, the biometric data is acquired by third sensor 50 by tracking sleeper S's heart rate variability such that the sleep recommendation provides insights into the sleeper S's stress levels and recovery ability. In some embodiments, the biometric data is acquired by tracking the sleeper's oxygen levels. In some embodiments, the biometric data is acquired by tracking the sleeper's surface temperature variations.

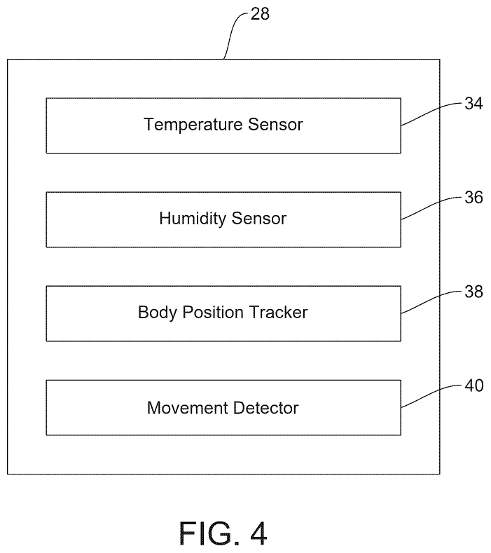

System 20 can include a receiver, such as, for example, an electronic device 52. In some embodiments, electronic device 52 is or includes a processor, such, as for example, a smartphone, as shown in FIG. 4. In other embodiments, electronic device 52 may be a desktop computer, tablet, smartwatch, etc. Electronic device 52 includes a processor that is configured to receive and analyze the data sets simultaneously and/or to receive and analyze the data sets from the first sensor 28 and/or second sensor 42 and biometric data from the third sensor 50 simultaneously. The processor is configured to execute an algorithm to provide a sleep product recommendation based on the simultaneously analyzed data sets and/or the simultaneously analyzed data sets and biometric data. In some embodiments, the algorithm can include an artificial intelligence algorithm. In some embodiments, electronic device 52 is configured to display information relating to the sleep product recommendation. That is, after data from first sensor 28, second sensor 42 and/or third sensor 50 are simultaneously analyzed and processed using the algorithm, the processor can cause electronic device 52 to display the sleep product recommendation.

As discussed herein, the differential data provided by the data sets and biometric data is used to generate the sleep product recommendation. In some embodiments, system 20 can be used to detect specific sources of discomfort, such as improper sleeper support. Accordingly, in such circumstances, the sleep product recommendation can suggest that sleeper S purchase bed and bedding having certain characteristics to provide proper support. That is, system 20 may be used to generate tailored recommendations for bed and bedding materials, including mattress type/firmness and/or pillow type/dimensions. For example, if system 20 were to detect inadequate support, the sleep product recommendation may suggest that sleeper S purchase a mattress and/or a pillow that is firmer than sleeper S's current mattress and/or pillow. Because system 20 utilizes a plurality of sensors that are used simultaneously to collect data and the data collected by sensors 28, 42, 50 is analyzed simultaneously by the processor to generate the sleep product recommendation, the sleep product recommendation can suggest bed and bedding based on numerous factors. Indeed, the suggestion can take into account data collected by first sensor 28, second sensor 42 and/or third sensor 50 to suggest bed and bedding tailored for sleeper S. Therefore, the sleep product recommendation can be based on microenvironmental factors from first sensor 28, macroenvironmental factors from second sensor 42 and/or biometric data from third sensor 50. As such, the bedding recommendation can suggest a mattress, for example, that includes an adjustable base or frame, based on the microenvironmental factors; that includes fans to move air within the mattress, based on the macroenvironmental factors; and/or that is soft and/or less dense than average, based on the biometric data.

In some embodiments, system 20 can be used to detect unsuitable environmental conditions. In such circumstances, the sleep product recommendation can also suggest that sleeper S alter the environmental conditions within area A. For example, the sleep product recommendation may suggest that sleeper increase or decrease the temperature and/or humidity within area A. Furthermore, after the data from first sensor 28, second sensor 42 and third sensor 50 are collected and analyzed simultaneously, the differential data provided by sensors 28, 42, 50 can be used to identify discrepancies between perceived and actual sleep behaviors, thus allowing the sleep product recommendation to suggest a change in sleep schedule, for example, that sleeper S may not be aware is causing sleep issues. Indeed, because system 20 utilizes a plurality of sensors that are used simultaneously to collect data and the data collected by sensors 28, 42, 50 is analyzed simultaneously by the processor to generate the sleep product recommendation, the sleep product recommendation can suggest altering environmental factors based on numerous factors. Indeed, the suggestion can take into account data collected by first sensor 28, second sensor 42 and/or third sensor 50 to suggest environmental changes. Therefore, the suggested environmental changes in the sleep product recommendation can be based on microenvironmental factors from first sensor 28, macroenvironmental factors from second sensor 42 and/or biometric data from third sensor 50. As such, the sleep product recommendation can suggest using a fan, based on the microenvironmental factors; can suggest using a humidifier, based on the macroenvironmental factors; and/or can suggest using an eye mask and/or blackout curtains, based on the biometric data.

The processor 52 algorithm may be structured to analyze the data set, the biometric data and subjective feedback separately or in aggregate. In other cases, the algorithm may assign weightings to various parameters to represent their relative importance in analyzing the captured information. The algorithm may also utilize the differential data from the first and second sensors 28, 42 rather than the discrete data for each. The algorithm may also utilize AI techniques and data to assist in analysis

The differential data provided by sensors 28, 42, 50 allows for the sleep product recommendation to be more accurate than if data collected from only one of sensors 28, 42, 50 was analyzed. Indeed, the differential data provided by sensors 28, 42, 50 allows multiple different conditions/parameters to be considered. For example, if only data collected by first sensor 28 were analyzed to generate the sleep product recommendation, the sleep product recommendation would not take into account conditions within area A (that can be acquired using second sensor 42) and/or biometric data of sleeper S (that can be acquired using third sensor 50). Likewise, if only data collected by second sensor 42 were analyzed to generate the sleep product recommendation, the sleep product recommendation would not take into account conditions within space 26 (that can be acquired using first sensor 28) and/or biometric data of sleeper S (that can be acquired using third sensor 50). Similarly, if only data collected by third sensor 50 were analyzed to generate the sleep product recommendation, the sleep product recommendation would not take into account conditions within space 26 (that can be acquired using first sensor 28) and/or conditions within area A (that can be acquired using second sensor 42).

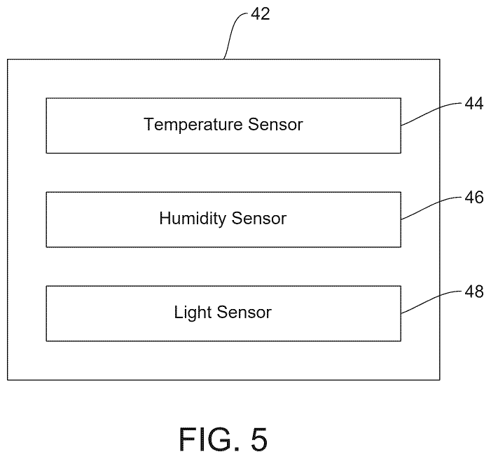

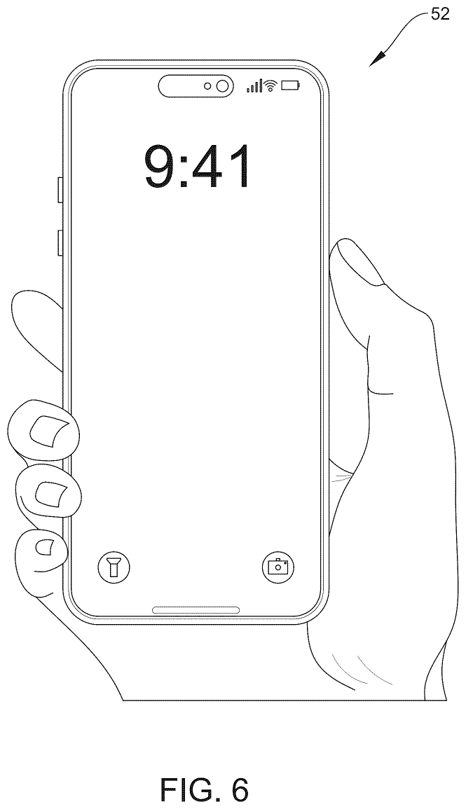

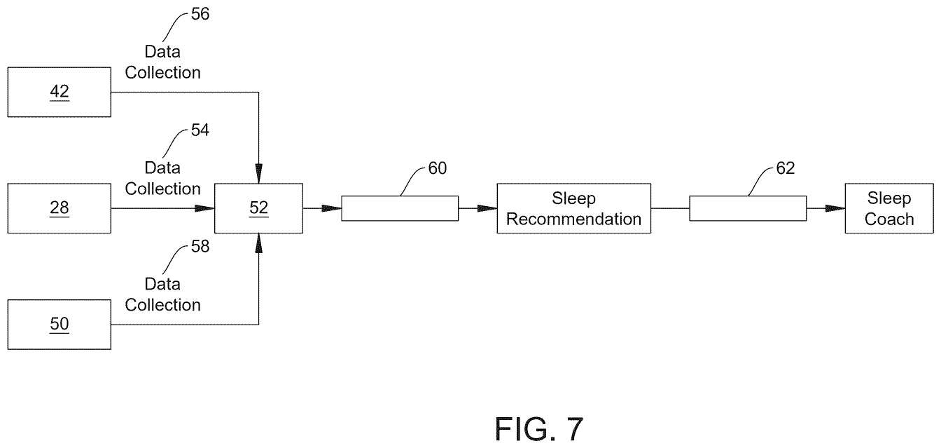

In operation and use, sleeper S positions body B on support S and then positions cover 24 over at least a portion of body B to create space 26. Sleeper S positions first sensor 28 on sleeper S's torso, back, or belly, as discussed above. Sleeper S positions third sensor 50 on sleeper S's wrist. In some embodiments, sleeper S may turn second sensor 42 from an off position to an on position. Sleeper S may then begin a sleep session (i.e., attempting to sleep, sleeping, and awakening). First sensor 26 collects data during the sleep session and sends the data collected by first sensor 28 to electronic device 52 in a step 54, as shown in FIG. 5. Second sensor 42 collects data during the sleep session and sends the data collected by second sensor 42 to electronic device 52 in a step 56. Third sensor 50 collects data during the sleep session and sends the data collected by third sensor 56 to electronic device 52 in a step 58. The processor of electronic device 52 aggregates and analyzes the data collected by sensors 28, 42, 50 via an algorithm in a step 60 to generate the sleep product recommendation, which can include bed and bedding suggestions and/or environmental changes, as discussed herein. Electronic device 52 can send the analytics or reports and a recommendation to a sleep advisor, the sleepers or other entity in a step 62. This allows the analytics and recommendation to be reviewed by sleeper S and/or the sleep advisor to organize a sleep plan and promote better sleep. In some embodiments, the sleep plan can include purchasing bed and bedding, such as, for example, mattresses and/or pillows, that were recommended in the recommendation. In some embodiments, the sleep plan can include altering area A, by altering the ambient temperature and/or humidity, for example, based on the recommendation.

It will be understood that various modifications may be made to the embodiments disclosed herein. For example, features of any one embodiment can be combined with features of any other embodiment. Therefore, the above description should not be construed as limiting, but merely as exemplification of the various embodiments. Those skilled in the art will envision other modifications within the scope and spirit of the claims appended hereto.

What is claimed is:

1. A system to promote sleep of a sleeper, the system comprising:
a first sensor configured to be coupled to the sleeper's body and to monitor a first set of sleep parameters during a sleep session to provide a first data set based on the first set of sleep parameters;
a second sensor that is spaced apart from the sleeper's body and is configured to monitor a second set of sleep parameters during a sleep session to provide a second data set based on the second set of sleep parameters; and
a processor configured to receive and analyze the data sets simultaneously and to execute an algorithm to provide a sleep product recommendation to a user based on the simultaneously analyzed data sets.

2. The system recited in claim 1, wherein the sleep product recommendation includes suggestions for sleep products based on the simultaneously analyzed data sets.

3. The system recited in claim 1, wherein analyzing the data sets simultaneously includes using differential measurements to compare differences between the data sets and determine the sleep product recommendation.

4. The system recited in claim 1, wherein the first set of sleep parameters includes movements of the sleeper during the sleep session and body positions of the sleeper during the sleep session.

5. The system recited in claim 4, wherein the movements record the frequency of the sleeper tossing and turning during the sleep session.

6. The system recited in claim 4, wherein the movements record the sleeper's movement patterns.

7. The system recited in claim 4, wherein the body positions monitor body positions assumed by the sleeper during the sleep session.

8. The system recited in claim 1, wherein the first set of sleep parameters includes localized temperature adjacent to the sleeper.

9. The system recited in claim 1, wherein the first set of sleep parameters includes localized humidity adjacent to the sleeper.

10. The system recited in claim 1, wherein the first sensor is configured to be coupled to at least one of the sleeper's torso, the sleeper's back and the sleeper's stomach.

11. The system recited in claim 1, wherein the second set of sleep parameters includes at least one of an ambient temperature, an ambient humidity and an ambient brightness.

12. The system recited in claim 1, wherein the processor compares the first data set with the second data set as part of analyzing the data sets.

13. The system recited in claim 1, wherein the processor generates and displays analytics in providing the sleep products recommendation.

14. The system recited in claim 1, wherein the processor is configured to receive subjective feedback from the sleeper in analyzing the data sets, and providing the sleep products recommendation.

15. A system to promote sleep of a sleeper, the system comprising:
a first sensor configured to be coupled to the sleeper's body and to monitor a first set of sleep parameters during a sleep session to provide a first data set based on the first set of sleep parameters;
a second sensor that is spaced apart from the sleeper's body and is configured to monitor a second set of sleep parameters during a sleep session to provide a second data set based on the second set of sleep parameters;
a third sensor configured to be coupled to the sleeper's body and to track biometric data pertaining to the sleeper during a sleep session; and
a processor configured to receive and analyze the data sets and the biometric data simultaneously and to execute an algorithm to provide a sleep product recommendation to a user based on the simultaneously analyzed data sets and biometric data.

16. The system recited in claim 15, wherein the sleep product recommendation includes suggestions for bedding based on the simultaneously analyzed data sets.

17. The system recited in claim 15, wherein analyzing the data sets simultaneously includes using differential measurements to compare differences between the data sets and determine the sleep product recommendation.

18. The system recited in claim 15, further comprising a covering that covers a portion of the support, the sleeper being configured for positioning in a space between the support and the covering, wherein the first set of sleep parameters includes localized conditions within the space.

19. The system recited in claim 15, wherein the localized conditions include fluctuations in temperature and/or humidity.

20. A method to promote sleep of a sleeper, the method comprising:
monitoring a first set of sleep parameters and providing a first data set based on the first set of sleep parameters;
monitoring a second set of sleep parameters and providing a second data set based on the second set of sleep parameters;
tracking biometric data pertaining to the sleeper; and
receiving and analyzing the data sets and the biometric data simultaneously and providing a sleep product recommendation to a user based on the simultaneously analyzed data sets and biometric data,
wherein the sleep product recommendation includes suggestions for bedding based on the simultaneously analyzed data sets,
wherein the bedding is selected from the group consisting of mattresses and pillows,
wherein analyzing the data sets simultaneously includes using differential measurements to compare differences between the data sets and determine the sleep product recommendation,
wherein the sleep product recommendation provides an accurate estimation of temperature and humidity conditions that are experienced by the sleeper during a sleep session,
wherein the sleep product recommendation includes suggestions for environmental adjustments, and
wherein the sleep product recommendation identifies discrepancies between perceived and actual sleep experiences.
